(19) United States
(12) Patent Application Publication
(10) Pub. No.: US 2008/0109027 A1
(43) Pub. Date: May 8, 2008

(54) METHOD AND SYSTEM FOR GASTRIC VOLUME CONTROL

(75) Inventors: Richard D.Y. Chen, Napa, CA (US); Christopher Scott Jones, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Fulfillium, Inc., Napa, CA (US)

(21) Appl. No.: 11/774,373

(22) Filed: Jul. 6, 2007

Related U.S. Application Data
(60) Provisional application No. 60/821,001, filed on Aug. 1, 2006.

(51) Int. Cl. A61M 29/00

(52) U.S. Cl. 606/191

(57) ABSTRACT

A gastric balloon includes a scaffold structure, one or more internal inflatable compartments within the scaffold structure, and one or more inflatable bladders formed over the space-filling compartment. The gastric balloon may be deployed transesophageally using a gastroscope and is inflated in situ, preferably using a combination of liquid and gas inflation media.

### CROSS-REFERENCES TO RELATED APPLICATIONS

The present application claims the benefit of provisional U.S. Application No. 60/821,001 (Attorney Docket No. 022209-000600US), filed Aug. 1, 2006, the full disclosure of which is incorporated herein by reference.

### BACKGROUND OF THE INVENTION

1. Field of the Invention

The present invention relates generally to medical apparatus and methods. More particularly, the present invention relates to apparatus and methods for expanding gastric balloons and other devices with minimum risk of accidental deflation.

Obesity is a serious medical condition and has become a widespread problem in the United States and many other industrialized countries. While many obese patients may be treated by modifications to diet and exercise, a number of morbidly obese patients are resistant to treatment and are candidates for surgical intervention. One surgical approach for treating morbid obesity is referred to as gastric or jejunoileal bypass where a major portion of the gastro-intestinal tract is surgically bypassed. While effective in some patients, gastric bypass procedures can have significant undesirable side affects. Moreover, the initial surgical procedure presents risks associated with open surgery. There are restrictive surgical procedures but they are less effective and still invasive. Consequently, an effective, non-invasive medical treatment with lower risks and minimal side effects is needed for many morbidly obese patients, who cannot tolerate surgical intervention, and most premorbidly obese patients, who have no effective treatment as their condition is not sufficiently severe to qualify them as surgical candidates.

As an alternative to such surgical procedures, the introduction of space-occupying structures into the stomach, often referred to as “gastric balloons,” has been proposed. Such gastric balloons may be introduced through the esophagus and inflated in situ in order to occupy a significant volume within the stomach.

Although found to be effective in some cases, the use of gastric balloons has been compromised by a number of deficiencies. The most serious is accidental deflation of the gastric balloon that can allow the balloon to pass the pyloric valve and enter the intestines. Such unintentional passage of the deflated balloon into the intestines can cause intestinal obstruction and be life-threatening. Consequently, gastric balloons currently marketed outside the US are generally indicated for use of only up to six months.

For these reasons, it would be desirable to provide improved gastric balloon structures and methods for in situ expansion of such balloons. The filled balloons should be inherently incapable of accidental deflation while permitting relatively easy intentional size reduction to permit removal. The gastric balloons should further resist passage into the pyloric valve and into the intestines, even when the balloon structure is compromised. In particular, it would be desirable for the device to maintain its “inflated shape” and size for a prolonged period of time even if the balloon wall has been punctured. Such a safety feature would extend the useful life of such devices and the treatment period in the product indication. It would be still further desirable if the features and structure of the balloon design were useful in other inflatable or expandable medical devices. At least some of these objectives will be met by the inventions described below.

2. Description of the Background Art

Gastric balloons and methods for their use in treating obesity are described in U.S. Pat. Nos. 6,746,460; 6,736,793; 6,733,512; 6,656,194; 6,579,301; 6,454,785; 5,993,473; 5,259,399; 5,234,454; 5,084,061; 4,908,011; 4,899,747; 4,739,758; 4,723,893; 4,694,827; 4,648,383; 4,607,618; 4,501,264; 4,485,805; 4,416,267; 4,246,893; 4,133,315; 3,055,371; and 3,046,988 and in the following publications: US2006/0282107; US 2006/0111777; US 2006/0111632; US 2005/0267596; US 2005/00267595; US 2004/0186503; US 2004/0186502; US 2004/0106899; US 2004/0059289; US 2003/0171768; US 2002/0055757; WO 03/095015; WO88/00027; WO87/00034; WO83/02888; EP 0103481; EP0246999; GB2090747; and GB2139902.

### BRIEF SUMMARY OF THE INVENTION

The present invention provides improved medical structures and methods for introducing those structures at a target location in a patient body. The medical structures will be expandable or “fillable,” where a filling material is introduced into the structure in situ in order to expand the structure to occupy space, to assume a desired geometry, to anchor the structure in place, or the like. The medical structures will comprise an expandable shell which may be introduced to the target location in an unexpanded (unfilled) configuration to simplify placement. The unexpanded shell is filled in situ with an elongate filling structure in a manner which expands the shell to assume a desired expanded configuration. An elongate filling structure may be formed continuously prior to introduction or may alternatively be introduced as a series of discrete segments or pieces which are assembled as they are being introduced and/or after they have entered an interior of the expandable shell. The elongate filling structure may be dimensionally stable and retain its shape after introduction to the shell. Alternatively, the shape of the elongate filling structure or a portion thereof may be modified as or after it is introduced into the expandable shell, where manipulation can be achieved through mechanical, chemical, physical, or other means. Optionally, the shell may have one or more interior component(s) which act to divide an interior volume of the shell into discrete regions, each of which are separately fillable by one or more elongate filling structure(s) or portion thereof. Alternatively, the interior component(s) may act to guide and/or displace the elongate filling structure as the filling structure is being introduced to the expandable shell. While the implantable medical structures of the present invention are particularly useful as gastric balloons for the treatment of obesity, they will have other uses as well, including use as breast implants, gastric banding implants, penile implants, and the like. While the remaining description herein is directed specifically at the construction and use of gastric balloons in accordance with the principles of the present invention, it will be appreciated that the broad principles of the present invention are applicable to other medical structures.

Gastric balloons in accordance with the principles of the present invention will typically have an overall volume or displacement selected to leave a residual volume in a proximal area of the stomach in the range from 10 ml to 100 ml, usually from 20 ml to 40 ml. As discussed in detail below, in some embodiments, the residual volume will be adjustable to optimize treatment on individual patients. The gastric balloons will typically be designed to conform to the natural shape of the gastric cavity while permitting the normal function of the stomach. The balloon will preferably have a crescent or “kidney” shape to align the balloon wall against the greater and lesser curvatures of the stomach and an oval cross section to conform to the shape of the cavity in the sagittal plane. Such a shape delineates a space proximally for the collection of ingested food and another space distally for active digestion.

The gastric balloons include an expandable shell and at least a first principal internal space-filling component, also referred to herein as the “elongate filling structure.” The elongate filling structure usually comprises a series of spaced-apart filling elements connected to each other to occupy space in an interior of the shell. The filling elements can be of any shape or size, and when connected together will usually be sufficiently large so that they cannot pass through the pylorus (even if the individual filling elements were small enough to pass through the pylorus). They can be connected to each other in various arrangements such as fastened to each other in an end-to-end manner, e.g., a “chain”, or fastened to another common object such as threaded on a tether, e.g., “string of pearls”, fastened to a chain, e.g., “bracelet of charms”, attached to or incorporated in a substrate, e.g., “tape of gumballs”. Alternatively, the exterior of the connected elements could form an even, continuous surface like a rope or cable having a circular or other cross-sectional shape. Such elongated arrangements facilitate both the introduction of the elements into the balloon and their extraction at the end of the treatment period. The elements may have individual characteristics which determine the compressibility and weight among other performance characteristics of the entire device.

The filling elements could be solid, hollow, porous, or small inflatable objects themselves. They can be made of any material, e.g., polymers, gels, metals, etc., that is biocompatible with implants in the human body but preferably of materials that are resistant to degradation in the gastric or other implanted environment and are durable to withstand peristalsis, regurgitation, and other natural forces. Optionally, the elements at each end of the elongate filling structure could be made more easily identifiable, i.e., of a different shape, size, color, material, radiopacity, magnetism, etc., in order to facilitate extraction or diagnostic imaging

The filling elements are typically introduced into the expandable shell of the balloon already connected in one or more continuous lengths or strands. Alternatively, individual elements can be introduced separately or in segments and connected inside the balloon. When connected end-to-end, the elements compose the elongated filling structure which has a length much greater than its width and which can be placed or arranged to conform to an interior region of the expandable shell. Alternatively, two or more elongate filling structures can be connected end-to-side or side-to-side to compose a larger, planar structure that can be arranged to the desired form or shape.

The filling elements are usually introduced into the expandable shell of the balloon in their final states, e.g. fully expanded, but still small enough to pass through the pharynx, esophagus, and gastro-esophageal junction. Alternatively, filling elements may be introduced into the shell of the balloon in a pre-deployed state, e.g., dehydrated, compressed, unexpanded, or partially expanded, etc. Once they have been placed inside the balloon shell, the such pre-deployed elements expand or are expanded to assume a space-filling configuration. For example, the elements could be expanded by inflation, filling, or absorption at least partly with one or more fluids, typically a gas such as air or a liquid such as a saline solution. Such filling or inflation of the space-filling elements will usually be accomplished from an external pressurized or other fluid source, but certain gaseous inflation media can be generated in situ within the element by chemical reactions induced by mixing reactants or otherwise initiating a gas-producing chemical reaction. Alternatively, the expansion can be a physical change such as using memory metals like nickel titanium alloy or switching from a constrained to an unconstrained mechanical state. Further alternatively, expansion could be induced by a change in temperature (e.g., cooled to body temperature) or by contact with the fluids inside the stomach (where the shell could be open or porous).

In the simplest embodiments of the present invention, the expandable shell will be substantially free from internal structure, and the elongate filling structure will assume a random configuration as it is introduced and folds upon itself to fill the shell. While certain elongate filling structures will be able to fold and conform to the geometry of the shell without substantial risk of tangling, other filling structures might become tangled and difficult to withdraw unless they are intentionally layered or structured as they are being introduced. Such layering or structuring can be accomplished by providing internal barrier structure(s) within the interior of the expandable shell, where the barrier structures are able to orient or direct the elongate filling structures as they are introduced into the shell. For example, fixed barriers can be placed within the expandable shell to divide the interior of the shell into two, three, four, or more separate compartments, where each of the compartments may be filled in order by the elongate filling structure(s), or alternatively may be separately filled by a plurality of discrete elongate filling structures. The barrier structure may also be movable relative to the expandable shell, where the movable barrier structure directs or orients the elongate filling structure as it is introduced. For example, the barrier structure may be a rotatable spool, where the elongate filling structure is wound or reeled over the spool to orient it as it is delivered into the expandable shell.

In some instances, the expandable shell may be expanded or filled only with the elongate filling structure. The remainder of the interior volume of the expandable shell may remain filled with air or may be unfilled so that the shell structure conforms tightly to the elongate filling structures. More usually, however, the expandable shell will be intentionally filled with a liquid and/or gas filling medium (either when the elongate filling structure(s) are introduced together or separately) in order to adjust the buoyancy of the gastric balloon and to controllably fill the remaining volume within the expandable shell.

The spaces in between the filling elements may be filled with compressible fluids (gases), incompressible fluids (liquids), or in some cases mixtures of gases and liquids. The use of a gas and liquid mixture for gastric balloon inflation has a number of advantages. A principal benefit is the ability to control buoyancy and weight distribution within the balloon, e.g., by filling most of the compartments with a gas and distributing the non-gas inflation medium in other compartments throughout the balloon, the risk of concentrated pressure points against the stomach is reduced. Second, by properly controlling the ratio of air or other gas to saline or other liquid, the gastric balloon can be provided with a desired buoyancy and mass within the stomach. Typically, the ratio of air:liquid can be in the range from 1:1 to 10:1, more preferably within the range from 3:1 to 6:1, depending on the total volume occupied by the device. Such ratios can provide effective densities relative to water at a specific gravity in the range from 0.09 to 0.5, usually from 0.17-0.33. Typically, the total weight of the filled balloon is in the range from 50 gm to 550 gm, usually being from 50 gm to 450 gm. The use of gastric balloons which are light and less dense will reduce the risk that the balloons will cause abrasion, pressure induced lesions, shearing lesions, or other trauma when implanted in the stomach for extended periods of time.

Optionally, a failure detection system may be provided in the gastric balloon. A chemical failure detection system may include a thin film or coating of a detectable substance, such as a dye, on the balloon so that the substance is released into the stomach in the event the integrity of the balloon is compromised and detected upon excretion or regurgitation by the patient. Optionally, different substances may be placed in the filling elements so that the particular element which failed may be identified based on what is detected. Optionally, the substance could be embedded in the wall of the balloon so that partial breach of the layer would result in the substance be in contact with the stomach contents. Incorporating the substance(s) in the device eliminates a step for the medical professional to measure and mix the substance(s) into the inflation media. Many errors including mixing ineffective concentrations such that detection becomes unreliable, contaminating the different components such that identification of the particular failed component becomes unreliable, confusing the substance(s) with its respective component, or simply forgetting to mix in the substance(s) is prevented. Furthermore, the detection mechanism is standardized for the device and easier for medical professionals other than the person deploying the device to diagnose any failure.

The balloon and the elongate filling structures may be delivered into the stomach via endoscopy. The delivered component typically has a relatively low-profile cylindrical shape, usually having a width or diameter no greater than about 30 mm, preferably no greater than about 20 mm, in order to permit delivery by itself or through a tubular introducer positioned through the esophagus. Once it is in place, the elongate filling structure can then be introduced into the balloon through a filling tube.

The expandable shell can be made from elastic and/or inelastic materials, such as silicone rubber and polyethylene terephthalate film (Mylar®), respectively′. As air can be a significant proportion of the filling medium, the device may be provided with compressibility to accommodate peristalsis.

Although the following inflation methods are described using the tethered array of space-filling elements, it will be appreciated that other configurations can be applied with similar principles.

Filling the expandable shell can be accomplished in a variety of ways. Most simply, the elongate filling structure will be pushed through a filling tube which is disposed in the esophagus and attached at a distal end to the expandable shell. In one instance, the filling structure could be manually pushed at the point it enters the filling tube until a major portion of its length has entered the expandable shell. The remaining length of the expandable structure could then be pushed in using a shaft, rod, or other pushing element. Alternatively, the pushing element could be used to engage a side of the elongate filling structure to advance discrete lengths of the filling structure through the tube. By using longer push tubes or other structures, the physician could extend the push tube into the expandable shell to help position, orient, rotate, or otherwise manipulate the elongate filling structure after it is entered the expandable shell.

By using a tether which is itself hollow, a guidewire or other elongate member, could be threaded through the lumen of the tether and used to push or pull the structure through the fill tube and into the balloon. Alternatively, the hollow tether could be inflated with a gas or liquid to stiffen the elongate filling structure to aid in pushing it into the expandable shell.

In other embodiments, the elongate filling structure may be propelled through a filling tube into the interior of the shell or a desired compartment thereof using a carrier fluid, e.g., gas, liquid or a mixture of the two, under external pressure. Any excessive fluid can be removed either by suction at the external end of the filling tube, by allowing overflow out of the shell into the gastric cavity, by allowing backflow of fluid through the filling tube, or by escape of the excess through another pressure relieving (vent) tube. Alternatively, the elongate filling structure may be drawn into the shell with suction through a second tube. This procedure is continued until the balloon is filled to the desired volume. When a carrier fluid is used, the fluid will also occupy the interior of the shell which may then be sealed.

In order to reduce the possibility of tangling and knotting of the elongate filling structure, certain embodiments of the present invention will include at least one “barrier structure” to control the filling pattern of the elongate filling structure. With a fixed internal barrier, the filling of multiple compartments may be accomplished in a variety of ways. Most simply, separate lengths of elongate filling structures may be propelled through a filling tube into the desired compartment using a carrier fluid, e.g., gas, liquid, or a mixture of the two, under external pressure. Any excessive fluid can be removed either by suction at the external end of the elongate filling tube, allowing backflow of fluid through the filling tube, allowing overflow out of the shell into the gastric cavity, or escape of the excess through another pressure relieving tube. Alternatively, the filling structures and carrier fluid, if any, may be drawn into the shell with suction through a second tube. This procedure is repeated until each compartment is filled. Alternatively, each filling structure may be drawn into the compartment via a guide wire or string. The guide wire or string is then cut and removed after the structure is in place. Again, this procedure is repeated with each filling structure until each compartment is filled.

With a spool barrier already attached to an interior wall of the shell, the shell may be expanded by filling with an externally pressurized fluid, e.g., gas, liquid, or a mixture of the two. The spool is then expanded in the same fashion or allowed to self-expand. The elongate filling structure(s) is/are drawn into the shell via a guide wire or string, and an end of the structure is fixed to the spool. The remainder of the elongate filling structure is then drawn into the shell by the rotation of the spool.

Alternatively, once an end of the filling structure is placed in the shell, an orifice where the strand enters the shell may be rotated such that the element is drawn in rings around the spool. This can be accomplished by propelling the filling structure(s) through a filling tube using a carrier fluid, e.g., gas, liquid, or a mixture of the two, under external pressure. Once the filling structure(s) are in place, excessive fluid can be removed either by suction at the external end of the filling tube, allowing overflow out of the shell into the gastric cavity, allowing backflow of fluid through the filling tube, or escape of the excess through another pressure relieving (vent) tube. Alternatively, the filling structure and carrier fluid, if any, may be drawn through the rotatable orifice into the balloon around the spool with suction through a second tube.

After the expandable shell is filled to a desired level with the filling structure or a combination of the filling structure and a filling fluid, the filling and pressure relieving tube, if any, are removed, and the filling orifice can be sealed in a variety of ways. For example, the orifice can be capped with a plug. Alternatively, a clip or ring can be secured around it. The orifice could also comprise a self-sealing valve which closes when the filling tube is disconnected. Closing the orifice would ensure containment of the filling structure(s) and filling media and prevent stomach contents from entering the balloon and contaminating the interior of the device

Optionally, the balloon may have compartments that do not communicate with each other with each compartment having its own filling orifice. Each such compartment can be thereby further divided into smaller compartments, optionally having internal barrier structures, where the compartments can be filled with elongate filling structure(s) and/or fluid(s) using the principles described above.

Not all interior volume(s) within an expandable shell need to be fully filled to achieve a desired size and shape. The remaining volume(s) may be filled at a later date with either a filling structure or any fluid, e.g., gas, liquid, or a mixture of both. This will enable the device to be refilled or adjusted for size in situ either for performance or comfort purposes.

At treatment termination or upon failure of the device, it will usually be necessary to completely remove the entire device. To effect such removal, the gastric balloon will again be accessed transesophageally, typically using a gastroscope. The gastric balloon will first be grasped or secured using a grasping tool. Then, one or more surfaces of the expandable shell may be penetrated and incised in order to release the liquid contents of the balloon into the stomach. Alternatively, the sealing component, if any, could be removed. The contents will be biocompatible gases or liquids so that release into the stomach will not be a concern. With the interior volume(s) of the balloon then exposed, an end or other attachment location of the elongate filling structure(s) will be identified and extracted by suction or by grasping and pulling it out of the balloon through the esophagus and out of the oral cavity. If the filling structure is connected in a planar or three dimensional fashion, the filling structure can be cut into single strands to facilitate extraction. After all the filling structure(s) have been extracted, the deflated shell may then be pulled through the patient's esophagus, typically by pulling with the grasping tool. It may be possible to pull the deflated gastric balloon through the working channel of the gastroscope, but more often the balloon will simply be withdrawn through the esophagus as the gastroscope is withdrawn.

In certain embodiments, all or some of the individual filling elements of the elongate filling structure could be made of a “digestible” material, that is a material which is dissolvable in the acidic environment of the gastric cavity. In that way, should any of the filling elements escape into the gastric cavity, they could dissolve or be digested, minimizing any risk to the patient.

Optionally, a sheath or other protective cover may be placed over elongate filling structures and the deflated balloon in order to reduce the risk of trauma or injury to the esophagus upon withdrawal.

Then the present invention optionally provides for failure detection. This is desirable and useful even for a single compartment balloon. For example, a substance may be disposed within any or all (at least one) of the internal volumes and/or the inflatable space-filling component(s), where the substance is detectable upon release and excretion or regurgitation by the patient. For example, the substance may be a dye, a scented composition, a benign symptom-inducing agent such as polyvinyl pyrolidine (PVP), or the like. The substance will usually be disposed within each of the interior volumes and the space-filling component(s) Optionally, different substances may be placed in different components so that the particular component which failed may be identified based on what is detected. The substance may be detectable directly by sight, smell, or sensation, and/or by reaction with water in the toilet optionally with the addition of a detection reagent. Particular detection systems and methods are described in co-pending, commonly-owned application Ser. Nos. 11/121,704 (US 2005/0267596; Attorney Docket No. 022209-000220US), filed on May 3, 2005; 11/170,274 (US 2006/0111777; Attorney Docket No. 022209-000400US), filed on Jun. 28, 2005; and 11/282,224 (US 2006/0111632; Attorney Docket No. 022209-000410 US), filed on Nov. 18, 2005, the full disclosures of which are incorporated herein by reference.

As a further option, at least a portion of the exterior of the inflatable balloon and/or the elongate filling structure may be coated or impregnated with an anti-microbial and/or adhesion resistant agent. Preferably, the entire exposed surface of all components of the balloon will be so coated or impregnated to inhibit colonization of the balloon by bacteria or other microbes, and/or reduce possible accumulation of food particles on the device. Suitable anti-microbial agents include polyethylenetetrafluoride (PTFE), and antibiotics. The balloon and/or the elongate filling structure could also be coated with hydrophilic coatings, lubricious coatings, and combinations thereof.

The present invention further provides methods for treating obesity in a patient. The methods comprise introducing at least an expandable shell of a gastric balloon structure to the patient's stomach. At least a portion of an expandable shell of the balloon is then filled at least partly with one or more elongate filling structure(s) with the remaining spaces (if any) optionally filled with compressible fluids, typically a gas such as air, nitrogen, or the like, and/or with an incompressible material, such as a liquid, gel, slurry, or the like. In this way, the buoyancy and compressibility of the balloon and/or the filling structure may be controlled within the limits described above.

The methods of the present invention may further comprise determining the size of the gastric cavity and selecting a gastric balloon of proper size prior to introducing the balloon to the stomach. Such size determination may comprise visually examining the gastric cavity, typically under direct observation using a gastroscope, but alternatively using fluoroscopy, ultrasound, x-ray or CAT scanning, or any other available imaging method. An estimate of the dimensions of the stomach and the size of the device can be made by direct observation of the interior of the stomach immediately prior to deployment. Alternatively, the dimensions of the feeding stomach, which is generally larger than the resting stomach, and the size of the device will be determined at an earlier session where the patient has consumed or swallowed a biocompatible filling medium, e.g., water, contrast medium, food, etc. A sufficient amount of filling medium will be consumed so that the imaging technique can detect full relaxation of the stomach during feeding and estimate its dimensions and size. After the stomach size has been determined, a balloon size will be selected to allow a predetermined residual volume, typically in the range from 400 ml to 1200 ml, preferably from 500 ml to 900 ml.

Introducing the gastric balloon may then comprise passing the expandable shell of the gastric balloon in an unexpanded configuration into the stomach through a gastroscope. Alternatively, the deflated balloon could be introduced into the gastric cavity via an attachment to an orogastric or nasogastric tube by itself or along the side of the gastroscope. The balloon will be oriented so that when filled, the balloon will open with curved geometry conforming to the curvature of the gastric cavity.

After the gastric balloon has been filled and optionally inflated, all associated filling tubes will be released, any other anchors or tethers attached to the balloon will be deflated, leaving the balloon free to “float” within the patient's stomach. By properly selecting the ratio of liquid inflation medium to gas inflation medium, as discussed above, the weight, distribution, and the buoyancy of the gastric balloon will be such that the balloon rests within the stomach without exerting undue pressure at any particular point, thus reducing the risk of abrasions or other trauma to the stomach lining. The inflated gastric balloon may be left in place for extended periods of time, typically as long as weeks, months, or even years.

After initial implantation has been completed, it may later become desirable to adjust the size and/or buoyancy of the balloon for purposes of patient comfort, efficacy, or other reasons. To perform such adjustments, the balloon will be transesophageally accessed, typically using a gastroscope with suitable working tools introduced therethrough. For example, the balloon may be grasped with graspers and inflation tubes may be suitably attached or docked to inflation ports on the balloon structure. Typically, the inflation ports will all be located near the end of the gastric balloon structure which is oriented toward the top of the stomach so that they are readily accessed through the gastroscope. After attachment with the inflation tube, the inflation medium can be introduced and/or extracted, depending on whether the particular component is to be enlarged, deflated, or have a buoyancy adjustment. Optionally, an incising instrument could be introduced through the gastroscope to penetrate and deflate any filled compartment to reduce the overall volume of the device and improve accommodation of the device. Typically, these compartments are small to allow minor adjustments without jeopardizing the integrity of the device itself. Optionally, a segment of the space-filling component can be excised to reduce the volume.

### BRIEF DESCRIPTION OF THE DRAWINGS

FIG. 1 is a side view of a gastric balloon constructed in accordance with the principles of the present invention, with a first space-filling component folded upon itself inside.

FIGS. 2A through 2F show alternative embodiments of a space-filling component constructed in accordance with the principles of the present invention.

FIGS. 3A through 3C are alternative cross-sectional views taken along line 3-3 in FIG. 1.

FIGS. 4A through 4C illustrate various methods for filling the balloon shell.

FIG. 5 illustrates an exemplary filling tube.

FIG. 6 shows the rotatable balloon orifice which selectively directs the placement of space-filling elements to individual compartments defined by a divider or around a spool.

FIG. 7 illustrates the filling of a balloon shell with a second component acting as a spinning spool.

### DETAILED DESCRIPTION OF THE INVENTION

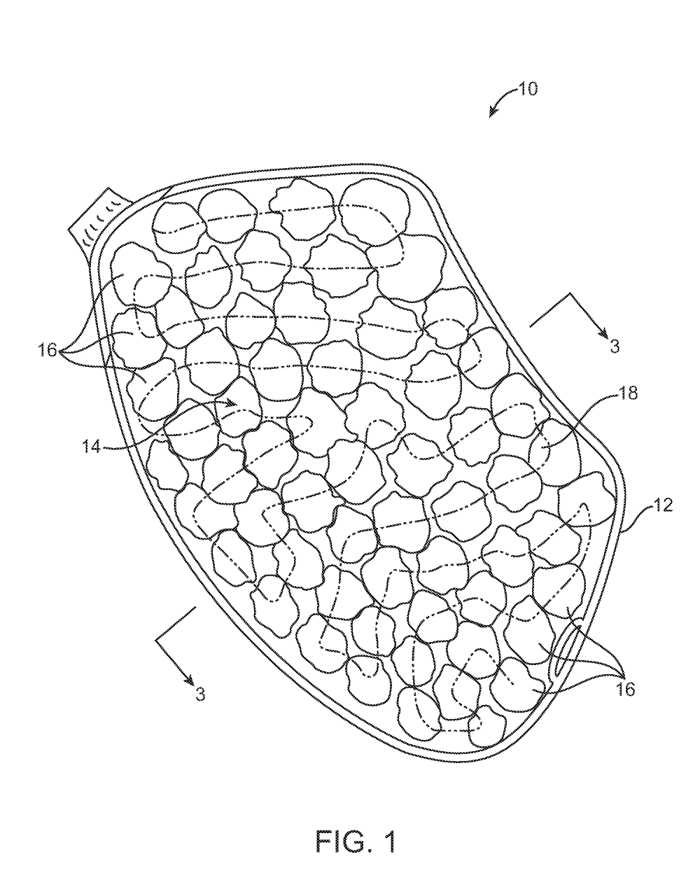

Referring now to FIG. 1, gastric balloon 10 comprises an expandable shell 12 having an elongate filling structure 14 therein. As shown in FIG. 1, the filling structure 14 comprises a plurality of space-filling elements 16 joined by a tether 18. The elongate filling structure 14 may have a variety of other configurations, and will typically have a continuous length of at least 10 cm, typically being in the range from 25 cm to 250 cm, and a maximum width no greater than 30 mm, typically being in the range from 10 mm to 20 mm. All dimensions are for the filling structure at the time it is introduced to the expandable shell. dimensions may vary after such introduction. Although usually comprising a plurality of discrete space-filling elements, the elongate filling structure could also comprise a monolithic molded or extruded polymer structure, for example being a long polymeric tube or cylindrical structure having dimensions in the range just set forth above, usually with a substantially constant outside diameter over its length. Optionally, the polymer could be a hydrogel or alternatively could be impermeable to water. Such monolithic polymeric structures could have either weights or gas-filled chambers formed therein to control buoyancy.

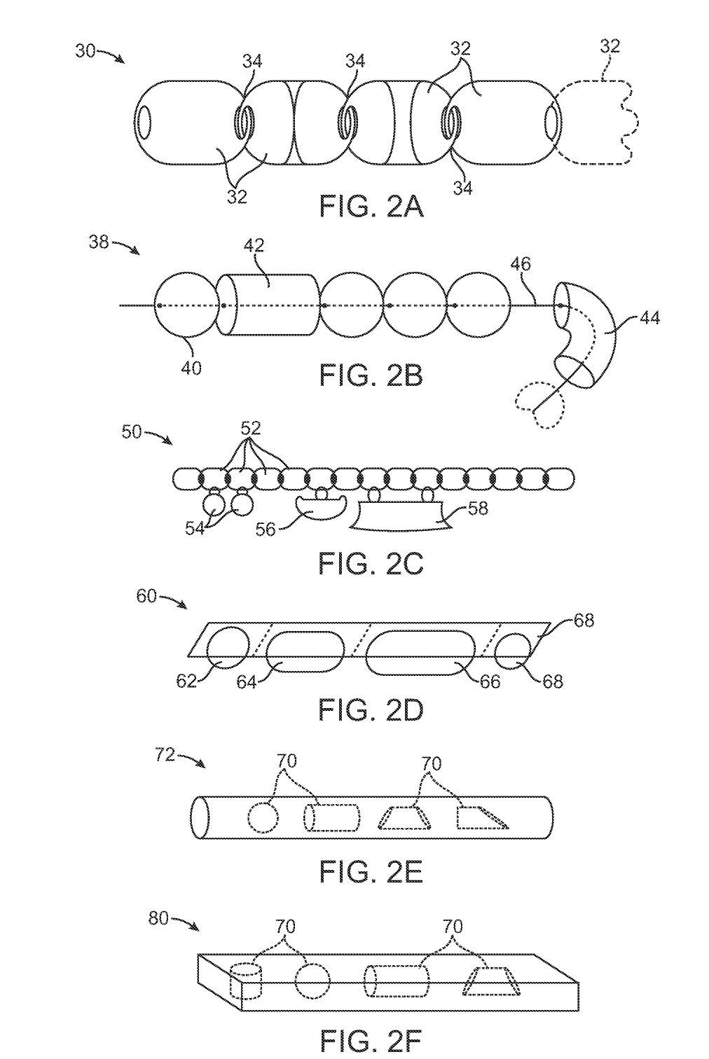

Referring now to FIGS. 2A-2F, other specific elongate filling structures constructed in accordance with the principles of the present invention will be described. In FIG. 2A, a chain 30 comprising individual links 32 which are joined together by clasps 34 is illustrated. Although four links are shown, it will be appreciated that the length of the chain 30 could be extended indefinitely (as shown in broken line).

As shown in FIG. 2B, an elongate filling structure 38 comprises a plurality of space-filling elements 40, 42, and 44 joined together on a tether 46 in a manner similar to a “string of pearls.” The shapes of certain of the individual space-filling elements 42 and 44 may be selected to provide a shape or structure to the resulting elongate structure when the individual elements are tightened onto tether 46. For example, the space-filling element 44 will tend to turn the structure to cause a series of folds in the structure as it is introduced to the expandable shell. The remaining elements 40 may be spherical, oval, or otherwise shaped, and will typically have no points or sharp edges which could cause abrasion and failure of the expandable shell and/or other filling elements.

An elongate filling structure 50 as shown in FIG. 2C comprises a plurality of individual space-filling elements 52 which may be joined by a tether (not shown) or by clasps (not shown) to form a linked structure as in FIG. 2A. Additional space-filling elements 54, 56, and 58 may be attached to the sides of the primary space-filling elements 52. The elongate filling structure 50 allows the selection of spaced-filling elements 54, 56, and 58 which may have different properties, such as density, shape, size, color, material, radiopacity, magnetism, or the like, which may be attached to the elongate filling structure which would itself have a uniform property, such as a uniform shape memory.

A further elongate filling structure 60 is illustrated in FIG. 2D, where individually shaped space-filling elements 62, 64, and 66 are attached to a tape-like backing 68. Alternatively, as shown in FIGS. 2E and 2F, individual space-filling elements 70 may be incorporated into a tubular structure 72 or a bar-like structure 80.

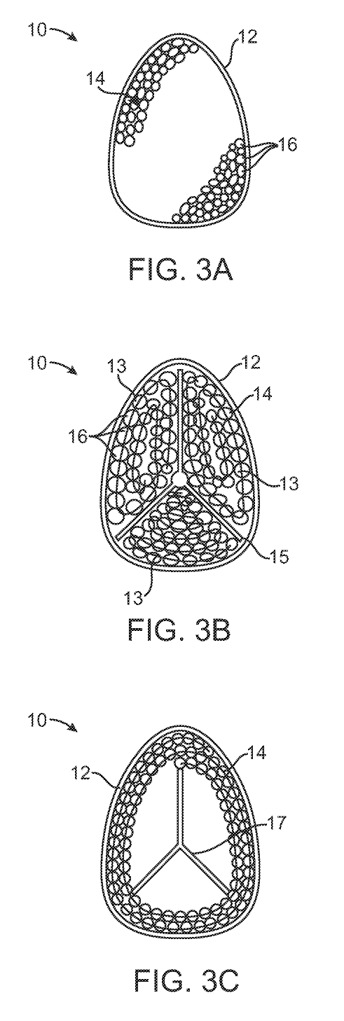

The elongate filling structures may be introduced into an interior of the expandable shell 12 of the gastric balloon 10 in a variety of ways, as shown in FIGS. 3A-3C. Most simply, the length of material 14 may be introduced into an interior of the shell 12 in a random manner without internal barriers or other structure, as shown in FIG. 3A. Alternatively, three separate lengths of the elongate filling structure 14 may be introduced into three separate compartments 13 defined by a fixed barrier structure 15 within the shell 12, as shown in FIG. 3B. Finally, as shown in FIG. 3C, the elongate space-filling structure may be wound around a rotatable internal barrier structure 17.

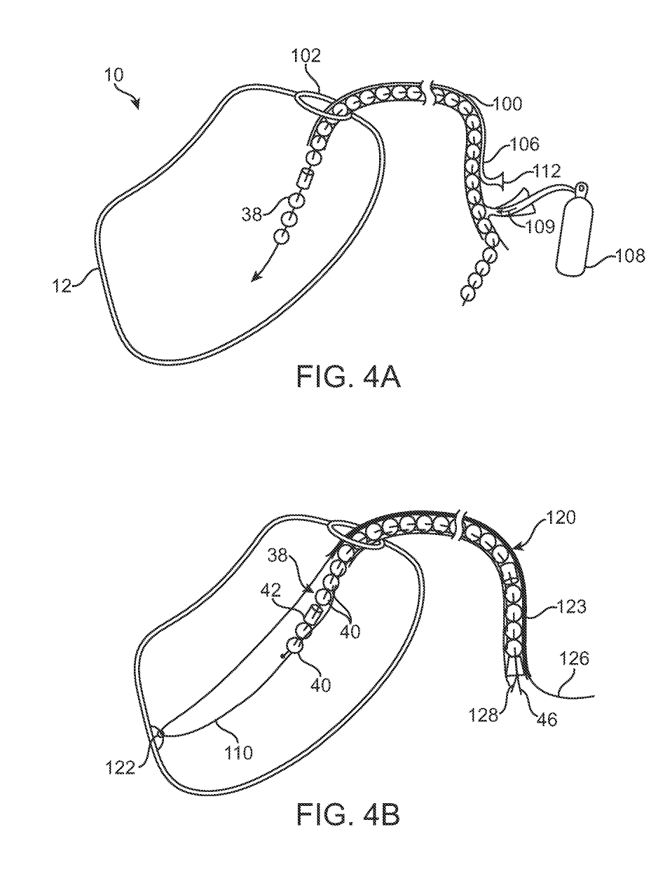

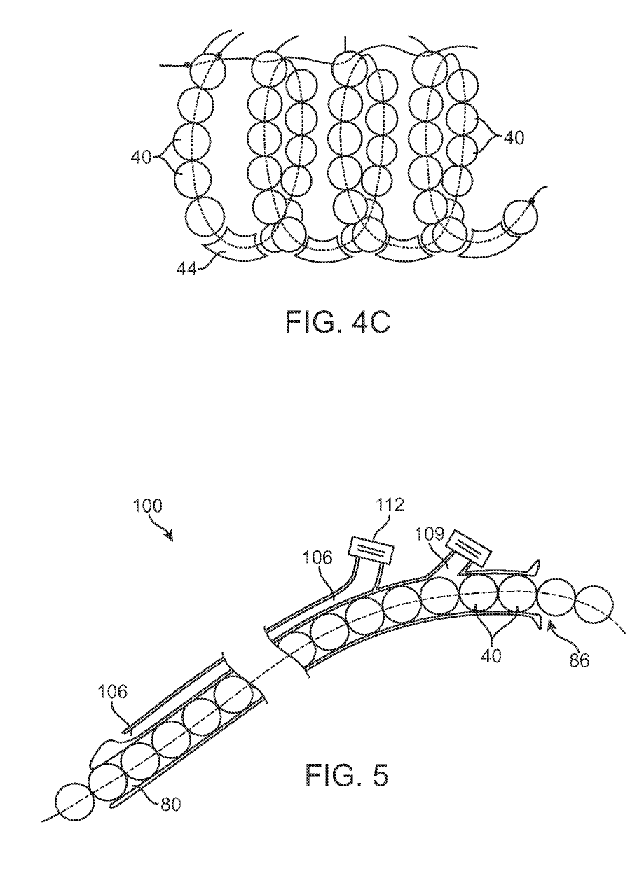

The elongate filling structures 14 may be introduced into the interior of the expandable shell 12 in a variety of ways, as shown in FIG. 4A. The elongate filling structure 38 is propelled through an introducing tube 100 attached to an opening or plug 102 at one end of the expandable shell 12 of the gastric balloon 10. The elongate filling structure 38 is propelled through a central lumen of the filling tube 100 by high pressure gas from a tank 104 connected to a port 109 on the proximal end of tube 100. Excess gas is vented through pressure relief lumen 106 and out of port 112.

As shown in FIG. 4B, space-filling elements 40 and 44 of the elongate filling structure 30 threaded on tether 46 are attached to a guide string 110. The elongate filling structure 38 passes through a filling tube 120, and the guide string 110 passes through a constriction 122 (shown as a hole inside the balloon) where the distal or leading end of the filling structure 38 will be positioned after deployment. The guide string 110 then passes out through a side lumen 123 in the filling tube 120 and has a free distal end 126. By pulling on the free distal end 126, a plug 128 at the proximal end of the elongate filling structure is advanced, pulling the entire string of space-filling elements 40 and 44 forwardly into the interior of the expandable shell 12. As shown in FIG. 4C, once inside the shell 12, the individual space-filling elements 40 and 44 will be drawn together, and the curved elements 44 will cause the structure to naturally coil or fold, as illustrated. When the entire elongate filling structure 38 is drawn within the shell 12, the proximal plug 128 may be removed and the guide string 110 withdrawn from the shell by pulling on the proximal end of string 46.

Referring now to FIG. 5, construction of the filling tube 100 is shown in more detail. The distal end of the filling tube 100 includes a main lumen 80 for passing the space-filling elements 40 and the vent lumen 106. The main lumen 80 terminates in a main port 86 for receiving the space-filling elements 40 and the side port 109 for receiving pressurized fluid from source 104. The vent lumen 106 terminates in a vent port 112.

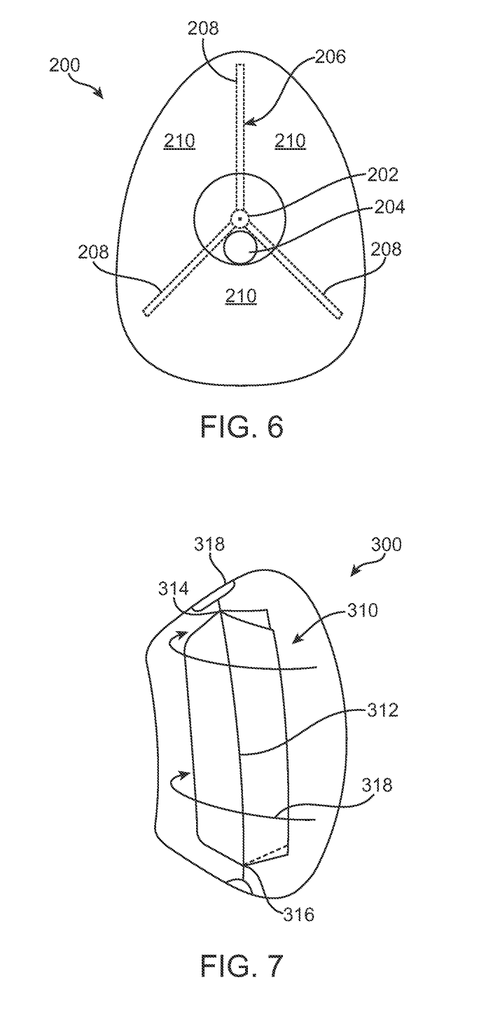

Referring now to FIG. 6, an expandable shell 200 includes a rotatable plate 202 having an offset orifice 204 therein. Fixed barrier structure 206 has three walls 208 for dividing the interior of the shell into three compartments 210. Plate 202 may be rotated to position the orifice 204 over each of the compartments 210 so that the elongate filling structure may be introduced sequentially into each compartment. Alternatively, a plate 202 may be rotated while the expandable shell 200 is being filled with a continuous filling structure so that the filling structure assumes the shape of a coil as it is laid over the spool formed by plates 208.

Referring now to FIG. 7, an expandable shell 300 has a rotatable filling structure 310 therein. The rotatable filling structure rotates about axis 312 which is rotatably attached at attachment points 314 and 316. The rotatable barrier 310 may be rotated in the direction of arrow 318 to act as a spool to draw in the elongate filling structure through port 318.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for implanting a medical structure, said method comprising:
introducing a shell into a target location in a body, and
filling the shell with at least one elongate filling structure.

2. A method as in claim 1, wherein the medical structure comprises a gastric balloon and introducing comprises placing an unexpanded balloon structure through the esophagus into a patient's stomach.

3. A method as in claim 2, wherein filling comprises passing the filling structure through a tubular introducer into the balloon structure, wherein the tubular introducer is disposed through the esophagus.

4. A method as in claim 1, wherein the medical structure comprises a breast implant, gastric band, or penile implant.

5. A method as in claim 1, wherein filling comprises passing a continuous length of the elongate filling structure into the expandable shell.

6. A method as in claim 5, wherein the entire length of the elongate filling structure is introduced as a single structure.

7. A method as in claim 1, wherein the elongate filling structure is introduced to the expandable shell in segments, wherein the segments are joined in situ into a continuous elongate filling structure.

8. A method as in claim 1, wherein the elongate filling structure comprises a generally uniform structure along most of its length.

9. A method as in claim 8, wherein the filling structure comprises a rope, cable, or tube.

10. A method as in claim 1, wherein the elongate filling structure comprises a plurality of space-filling elements connected in a linear array.

11. A method as in claim 1, wherein a barrier structure is disposed inside the shell prior to filling the shell with the elongate filling structure.

12. A method as in claim 11, wherein the barrier structure is preformed within the shell.

13. A method as in claim 12, further comprising deploying the barrier structure after the shell has been introduced to the target location.

14. A method as in claim 1, wherein the barrier is fixed within the shell to define different compartments which are filled with at least one elongate filling structure.

15. A method as in claim 1, wherein the barrier structure comprises a movable spool which receives the elongate filling structure(s).

16. An implantable system comprising:
shell which can be introduced in a non-expanded configuration to a target location in a body; and
at least one elongate filling structure which may be introduced to an interior of the shell to fill the shell.

17. A system as in claim 16, wherein the shell is shaped as a gastric balloon, a breast implant, a gastric band, or a penile implant.

18. A system as in claim 17, wherein the gastric balloon shall has an arcuate profile.

19. A system as in claim 16, wherein the shell is inelastic.

20. A system as in claim 16, wherein at least a portion of the shell is elastic.

21. A system as in claim 16, wherein the elongate filling structure comprises a continuous length of material having a length of at least 10 cm and a maximum width of less than or equal to 30 mm.

22. A system as in claim 16, wherein the continuous length of material is a monolithic molded or extruded polymer structure.

23. A system as in claim 16, wherein the elongate filling structure comprises a plurality of space-filling elements connected in a linear array.

24. A system as in claim 23, wherein the space-filling elements are arranged in tandem on a tether.

25. A system as in claim 23, wherein the space-filling elements are linked together end-to-end.

26. A system as in claim 23, wherein the space-filling elements are solid.

27. A system as in claim 23, wherein the space-filling elements are filled with a fluid.
